(19) United States
(12) Patent Application Publication
(10) Pub. No.: US 2005/0064014 A1
(43) Pub. Date: Mar. 24, 2005

(54) Use of cystathionine

(76) Inventors: Paul-Andre Finot, St-Legier (CH); Tuong Huynh-Ba, Pully (CH); Jacques Vuichoud, La Tour-De Peilz (CH); Denis Breuille, Epalinges (CH)

Correspondence Address: BELL, BOYD & LLOYD LLC, P. O. BOX 1135, CHICAGO, IL, 60690-1135, US

(21) Appl. No.: 10/492,739

(22) PCT Filed: Oct. 8, 2002
(86) PCT No.: PCT/EP02/11266

(30) Foreign Application Priority Data
Oct. 16, 2001 (EP) 0120390.2

(51) Int. Cl. A61K 47/00; A61K 31/198

(52) U.S. Cl. 424/439; 514/562

(57) ABSTRACT

The present invention relates to the use of cystathionine in a nutritional composition, a food supplement, a prophylactic composition or an infant formula.

### BACKGROUND OF THE INVENTION

The present invention relates to a nutritional composition, to a food supplement, to a prophylactic composition and to an infant formula containing cystathionine.

Patent application EP 0655244 A1 describes an amino acid-based nutritional composition for treating infections, containing a proportion of pharmacologically active cysteine greater than the proportion of cysteine corresponding to the needs of a normal individual. However, free cysteine has major disadvantages: firstly, it is poorly resistant to thermal treatments since it oxidizes, in particular to cystine; secondly, cysteine has poor organoleptic qualities since, in weakly giving off hydrogen sulfide and other sulfur-containing compounds, it engenders an odor and also a very unpleasant taste in the nutritional solution.

Given these difficulties in enriching a product in cysteine due to its instability, other solutions must be found for enriching nutrition products, for example using cysteine precursors. Methionine is an essential amino acid which allows the biosynthesis of cysteine in a succession of 5 steps involving the synthesis of homocysteine (in 3 steps) and then that of cystathionine in a step which is irreversible, then finally to cysteine. We have shown that providing methionine does not make it possible to induce the beneficial effects observed by enriching the diet in cysteine. An object of the present invention is therefore to provide cystathionine since, firstly, it is directly converted to cysteine and, secondly, we have shown that cystathionine is much more stable than cysteine.

Cysteine is one of the 20 amino acids which are found in the human body. It is a nonessential amino acid, which means that the human body is capable of synthesizing it. However, it is important since it can be converted into many compounds. Cysteine is oxidized to cysteine sulfinic acid, and then, by decarboxylation, to hypotaurine, which gives taurine. Also by decarboxylation, it can result in the synthesis of acetyl CoA and can, furthermore, be converted into pyruvate. As a sulfur-containing amino acid, cysteine allows the formation of disulfide bonds which serve to stabilize the three-dimensional structures of extracellular circulating, intracellular and membrane-bound proteins. Furthermore, the supply of cysteine is important in cases where stress is observed in the organism, since it is found in high proportion in the proteins synthesized in response to inflammatory situations (whether acute or chronic), and is one of the constituents of glutathione.

Glutathione is a tripeptide made up of glycine, glutamic acid and cysteine. It acts as a coenzyme in living cells, and is involved in redox reactions and in amino acid transport. It is also and especially one of the most important antioxidants in the body, which neutralizes free radicals and thus prevents the risks of cancer and of aging. Finally, glutathione is a major detoxifying agent in the liver and thus contributes to the elimination of xenobiotics.

Scientific studies have been carried out on cystathionine in vitro, on isolated cells, and in vivo. As early as 1942, the article by Vigneaud et al. (J. Biol. Chem., 143, 59, 1942) mentions the possibility that cystathionine is metabolized to cysteine in mammalian tissues. Since the article by Hope (Proc. Intern. Congr. Biochem. 4th, Vienna, 1958, 13, 63, 1960), it has been well known that cystathionine is an intermediate molecule in trans-sulfidation, which naturally transfers the sulfur of methionine, via homocysteine, to give cysteine. P. W. Beatty and D. J. Reed (Arch. Biochem. Biophys., 1980, 204(1), 80-87) mention, for their part, the involvement of cystathionine in glutathione biosynthesis. Finally, very recently, the article by MacCoss et al. (Am. J. Physiol. Endocrinol. Metab, 2001, 280, E947-E955) measures the plasma enrichment of cystathionine after having infused labeled methionine, thus quantifying, in humans, the rate of conversion of methionine to cystathionine.

The present invention proposes to disregard these inconveniences by enriching the nutritional compositions in cystathionine in order to provide cysteine and to maintain the glutathione status in the consumer's or patient's body.

### SUMMARY OF THE INVENTION

The present invention relates to a nutritional composition comprising cystathionine for oral, enteral or parenteral nutrition, to a food supplement comprising cystathionine, to a prophylactic composition comprising cystathionine, or to an infant formula comprising cystathionine. Methods of treating a variety of conditions utilizing cystathionine are also provided. In addition, methods of making compositions including cystathionine are also provided.

Additional features and advantages of the present invention are described in, and will be apparent from, the following Detailed Description of the Invention.

### DETAILED DESCRIPTION OF THE INVENTION

As mentioned above, cysteine has major disadvantages both from the point of view of its heat-stability and from the organoleptic point of view. Surprisingly, cystathionine does not exhibit these negative aspects. Cystathionine is a product of condensation between homocysteine and serine, and it is converted to cysteine by the action of cystathionase. This dipeptide of interest is naturally present in mammalian tissues.

A first aspect of the invention concerns a nutritional composition comprising cystathionine, administered orally, enterally or parenterally. A second aspect of the invention concerns the food supplements containing cystathionine. A third aspect of the invention concerns the prophylactic compositions containing cystathionine. A fourth aspect of the invention concerns the infant formulae containing cystathionine. A final aspect of the invention concerns the use of cystathionine.

The term “cystathionine” is intended to mean cystathionine in the L form, cystathionine in the D form, cystathionine salts or a mixture of various forms of cystathionine.

The term “nutritional composition” is intended to mean any composition for oral, enteral, parenteral, culinary or dietary nutrition, inter alia, intended for humans or for animals. The term “enteral nutrition product” is intended to mean any type of composition which can be introduced artificially into the organism by the digestive tract. The term “parenteral nutrition product” is intended to mean any composition (medicinal or nutritive, for example) which can be introduced into the organism via a route other than the digestive tract: in particular by subcutaneous or intramuscular injection, by peritoneal injection, or by intravenous infusion, inter alia. The term “food composition” is intended to mean any liquid, viscous, structured, gelatinous, solid, iced, refrigerated or powdered composition, or any composition which has undergone a manufactured conversion in particular. It may be possible to conserve the nutritional composition according to the present invention at ambient temperature, refrigerated, frozen, dehydrated; it may in particular be liquids, drinks, soup, mousselines or purees, cereals, mayonnaise, salad dressing, pastry or pizza dough, pasta, deep-frozen products, dessert creams, dairy products, or else coffee powder, powdered cocoa, inter alia.

The term “food supplement” is intended to mean any supply of nutrients intended to improve the quality of the diet of a given human or animal population, which may or may not be normal, and any food additive. This definition also comprises any substance intentionally added to a foodstuff, generally in small amounts, so as to facilitate the conversion thereof, to stabilize or improve its qualities, its texture, its taste or its appearance, or to facilitate its preparation. The term “food additive” is intended to mean any substance, the use of which results or may probably result in said substance or its byproducts becoming an integral part of a food or modifying the specific characteristics of this food.

The food supplement as defined in the present invention may be in the form of a powder, a gel, a liquid, an emulsion, tablets or any other form which is ingestible with or without liquid.

For example, the cystathionine according to the present invention could be incorporated into a food supplement also comprising vitamins and/or minerals.

The term “prophylactic composition” is intended to mean any means intended to prevent the appearance of diseases, and also any agent used in a protective or preventive capacity against a disease. The prophylactic composition according to the present invention can be used in humans or animals, it being possible for the latter to be bred industrially or on a small scale. It concerns, in particular, poultry, rabbit, sheep, pig, calf, rodent or alternatively fish farming.

The prophylactic composition according to the present invention can, besides cystathionine, contain other amino acids for example. It can be a complete nutritional composition, a food supplement or an injectable product.

The term “infant formula” is intended to mean any substitute or complement for maternal milk intended for a child under the age of 12 months, covering the normal nutritional needs, and also any modifiable standard for children having specific nutritional needs, and the successive formulae. The term “successive formula” is intended to mean any foodstuff constituting part of the weaning diet from the age of 6 months to the age of 36 months.

The cystathionine content in the nutritional composition according to one of the aspects of the invention is from 0.01 to 100 g per liter or per kilo of food composition. Preferably, the composition according to the invention contains between 0.1 and 50 g of cystathionine per liter or 0.01 to 150 g per kilo of food composition.

The remainder of the composition can contain any type of ingredients and of excipients, in particular protein sources (whole proteins, peptides and/or free amino acids), lipid sources, in particular fatty acids which modify the inflammatory response, carbohydrate sources, sources of soluble and/or insoluble fiber, vitamins (in particular antioxidant vitamins), micronutrients, colorants, flavorings, stabilizers, etc. It can also contain growth factors, bacteria and/or their metabolites, flavonoids or carotenoids, including lycopene or lutein, for example. Finally, it can contain pharmaceutical molecules intended to act against any type of disease or of syndrome, in particular oxidative stress, hypermetabolism, cancer or cardiovascular diseases, for example.

The cystathionine content in the food supplement according to a second aspect of the invention is between 0.1 and 100% of said supplement (percentage by weight of solid), and even more optimally between 1 and 50% by weight. The remainder of the supplement can contain any type of excipient which can be used in the agrofoods industry, in particular carbohydrates, proteins, flavorings or colorants. It can also contain other food supplements such as vitamins, free amino acids or peptides, pharmaceutical molecules, acidifying agents, thickeners, sweetening agents or sweeteners, antioxidants, milk or milk proteins, fruit or vegetable juices, flavorings, for example.

The cystathionine content in the prophylactic composition according to a third aspect of the invention is between 0.1 and 99% of said composition (percentage by weight of solids), and even more optimally between 30 and 60% by weight. The remainder of the composition can contain any type of nutrient and of excipient which can be used in the agrofoods industry, in particular sugars, proteins, flavorings or colorants. It can also contain other food supplements such as vitamins, amino acids, antibiotics, enzymes or pharmaceutical molecules, for example.

The cystathionine content in the infant formula according to a fourth aspect of the invention is between 0.1 and 99% of said formula (percentage by weight of solids), and even more optimally between 0.1 and 25% by weight. The remainder of the formula can contain any type of nutrient or of foodstuff generally used in the agrofoods industry, and particularly in the baby food industry, in particular carbohydrates, proteins, flavorings or colorants. It can also contain other food supplements such as vitamins, free amino acids or peptides, pharmaceutical molecules, acidifying agents, thickeners, sweetening agents or sweeteners, antioxidants, milk or milk proteins, fruit or vegetable juices, flavorings, for example.

The aim of the use of the cystathionine according to the invention is to provide cysteine, so as to maintain the glutathione status and to allow synthesis of the cysteine-rich proteins synthesized in certain pathologies. It can also be used in sports nutrition, to prepare for exercise, during exercise, but also during the recovery phase which follows exercise.

The use of cystathionine for facilitating the endogenous synthesis of glutathione can also occur under situations of oxidative stress, of hypermetabolism, of infection or of inflammation, of respiratory stress or of muscle loss. It can also be added to the diet of individuals who have suffered burns or who have substantial wounds, of premature infants or infants with stunted growth.

The cystathionine contained in a composition according to any one of the aspects of the invention can be combined with hydrocolloids or with fiber, for example, which makes it possible to overcome its poor solubility. The combined fiber may, in particular, be gum arabic, xanthans or gum accacia. Other means for increasing the solubility of the cystathionine and for increasing the viscosity of the solution containing the cystathionine can be used, even though they are in no way essential for implementing the present invention. They may, inter alia, be means such as encapsulation or the use of double emulsions.

By way of example and not limitation, examples of the present invention will now be given.

Tests Concerning Physicochemical and Organoleptic Behaviors

Cystathionine is found to be a very stable amino acid. It was tested while added to SONDALIS (trademark), and compared to cysteine in equivalent molar concentration (41 mM, i.e. 5 g of cysteine/L). SONDALIS is an enteral nutrition product comprising 35% of the energy in the form of lipids, 50% in the form of carbohydrates, in particular of maltodextrins, and 15% in the form of proteins, and also vitamins, including vitamins A, B6, B12, C, D, E and K, and mineral salts such as phosphorus and magnesium, inter alia. 1.0 mL of supplemented SONDALIS was brought to 121° C. for 15 minutes in an autoclave. This represents a strong heat treatment, the procedure having lasted more than two hours from the beginning of the handling to the temperature reduction phase. These harsh conditions are confirmed by a thick consistency and a brown color obtained on the product.

The comparative results appear in the table below.

| Solution tested | Additional odor |
|---|---|
| SONDALIS alone | No additional odor |
| SONDALIS + L cystathionine | No additional odor |
| SONDALIS + L cysteine | Sulfur-based additional odor |

The absence of odor in the cystathionine-supplemented product is a first important element which proves its compatibility with an oral formula, in particular, insofar as the oxidation and decomposition of compounds containing sulfur produce derivatives with a strong unpleasant odor.

Test Concerning Stability

The stability of the cystathionine compared to that of cysteine was tested with deionized water and in various liquid enteral formulae (SONDALIS and REABILAN). REABILAN and SONDALIS (registered trademarks) are different enteral compositions comprising 35% of energy in the form of lipids, 50-52.5% in the form of carbohydrates, in particular of maltodextrins and starches, 12.5-15% in the form of peptides or proteins, and also comprising vitamins, mineral salts and other compounds such as taurine or L-camitine.

Stability in Water:

The cystathionine was added at its maximum level of solubility in water at 25° C., i.e. 0.6 mg/mL of water. The mixture was treated at 121° C. for 15 minutes in an autoclave and was analyzed.

In water, cystathionine is very stable, even after treatment at 121° C. for 15 minutes, since the degree of recovery is 100%, and the cysteine is slightly oxidized to cystine (6%).

Stability in the Enteral Formulae

Stability Under Harsh Sterilization Conditions (121° C.-15 min) at 3 mM Concentration

In the enteral formulae, and before heat treatment, the cysteine is relatively unstable: it is highly oxidized and can even be destroyed, according in particular to the amount added. Unlike cysteine, the cystathionine is found to be very stable.

In the enteral formulae, and after heat treatment, the amount of available cysteine decreases very substantially, and the relative loss of cysteine increases when the amount added decreases. The stability of cysteine and that of cystathionine depend on the product to which they are added; however, and whatever the handling conditions tested, the cystathionine is always more stable than the cysteine.

The results obtained after heat treatment (121° C. for 15 minutes in an autoclave) are shown in the table below:

| | L-cystathionine | | | | |
|---|---|---|---|---|---|
| | (3 mM) | | | | |
| | Non- | | L-cysteine (3 mM) | | |
| | degraded | Evaluated | Free | Oxidized | Evaluated |
| | form | loss | cysteine | cysteine | loss |
| SONDALIS | 83% | 17% | 11% | 3% | 86% |
| REABILAN | 100% | 0% | 12% | 5% | 83% |

Unlike cysteine, when cystathionine is added to an enteral solution, there is no release of H₂S since there is no release of a sulfur-based odor. Cystathionine therefore exhibits organoleptic qualities which are very largely superior to those of cysteine.

Stability Under THA Sterilization Conditions (140° C.-15″), at High Concentration (43 mM) in SONDALIS

The THA (tubular heating apparatus) sterilization conditions correspond to UHT conditions even though they do not exactly reproduce the UHT (ultra high temperature) conditions often used in an industrial process. The THA conditions therefore conventionally induce much less degradation than those described above, but slightly more than industrial UHT conditions.

The high concentration (43 mM) was chosen because it falls within the average of that which may be recommended in enteral nutrition products.

Under these conditions, the losses of cysteine are lower than for the harsh sterilization conditions (121° C.-15′) but remain much too high (27%) for use in a nutrition product. On the other hand, the stability of the cystathionine is very good since the evaluated losses are at the limit of reproducibility of the assaying method.

| | L-cystathionine | | | | |
|---|---|---|---|---|---|
| | (43 mM) | | | | |
| | Non- | | L-cysteine (43 mM) | | |
| | degraded | Evaluated | Free | Oxidized | Evaluated |
| | form | loss | cysteine | cysteine | loss |
| SONDALIS | 96% | 4% | 66% | 7% | 27% |

The following examples are given only by way of illustration.

### EXAMPLES

### Example 1

### Enteral Nutrition Product

An enteral nutrition product with an energy value of 1000 kcal/L is prepared, containing in aqueous phase 38 g/L of proteins consisting of a mixture of casein and soybean proteins, 35 g/L of a mixture of lipids comprising medium-chain triglycerides, corn oil, soybean oil, and rapeseed oil, and 125 g/L of carbohydrates in the form of maltodextrins. It also comprises the compounds mentioned in the table below (in which the word “vitamin” is abbreviated to “Vita”, “RE” means “retinol equivalent” and “TE” means “tocopherol equivalent).

The proteins also contain 10 g/L of cystathionine.

| Compound | Unit | Value | Compound | Unit | Value |
|---|---|---|---|---|---|
| Vita A | μg/L RE | 450 | Vita B6 | mg/L | 1.4 |
| Vita E | mg/L TE | 10 | Pantothenic acid | | 5 |
| Vita D | μg/L | 2.5 | Choline | | 200 |
| Vita K | | 30 | Sodium | | 600 |
| Folic acid | | 180 | Potassium | | 1200 |
| Vita B12 | | 3 | Chloride | | 1100 |
| Biotin | | 100 | Calcium | | 500 |
| Iodine | | 100 | Phosphorus | | 530 |
| Selenium | | 44 | Magnesium | | 200 |
| Chromium | | 70 | Manganese | | 2 |
| Molybdenum | | 75 | Iron | | 10 |
| Vita C | mg/L | 53 | Copper | | 1 |
| Thiamine | | 1.0 | Zinc | | 10 |
| Riboflavin | | 1.2 | Fluoride | | 1 |
| Niacin | | 12 | | | |

### Example 2

### Food Supplement

A food supplement intended for elderly individuals, containing 100 kcal per 100 mL of supplement in liquid form, is prepared. The final composition of the product is as follows:

Nutritional composition

| | | |
|---|---|---|
| Proteins | 3.8 | g |
| Carbohydrates | 13.8 | g |
| Fats | 3.3 | g |
| Fibers | <0.5 | g |

Minerals

| | | |
|---|---|---|
| Sodium | 0.035 | g |
| Potassium | 130 | mg |
| Calcium | 65 | mg |
| Phosphorus | 65 | mg |
| Magnesium | 12 | mg |
| Chloride | 70 | mg |
| Iron | 0.33 | mg |
| Zinc | 0.75 | mg |
| Copper | 0.10 | mg |
| Manganese | 0.20 | mg |

Vitamins

| | | |
|---|---|---|
| Vitamin A | 80 | μg (i.u.) |
| Vitamin D | 0.50 | μg (i.u.) |
| Vitamin E | 2.0 | mg |
| Vitamin K | 5.0 | μg |
| Vitamin C | 10 | mg |
| Vitamin B1 | 0.15 | mg |
| Vitamin B2 | 0.20 | mg |
| Pantothenic acid | 0.63 | mg |
| Vitamin B6 | 0.20 | mg |
| Vitamin B12 | 0.50 | μg |
| Niacin | 2.0 | mg |
| Folic acid | 25 | μg |
| Biotin | 0.015 | mg |

The proteins used in this example originate from powdered skimmed milk, from whey, and from powdered egg yolk.

The food supplement described in this example contains 15 g of cystathionine.

### Example 3

### Infant Formula

An infant formula is prepared by mixing the following ingredients in the proportions indicated. The final product contains 519 kcal per 100 g, and is in the form of a powder.

| | | |
|---|---|---|
| Fat | 27.7 | g |
| Milk fat | 0.7 | g |
| Mixture of fats (150) | 26.8 | g |
| Lecithin | 0.2 | g |
| Linoleic acid | 4.1 | g |
| α-Linolenic acid | 525 | mg |
| Proteins | 9.5 | g |
| Available carbohydrates | 57.9 | g |
| Lactose | 57.9 | g |
| Minerals (ash) | 1.9 | g |
| Sodium | 120 | mg |
| Potassium | 460 | mg |
| Chloride | 330 | mg |
| Calcium | 320 | mg |
| Phosphorus | 160 | mg |
| Magnesium | 36 | mg |
| Manganese | 40 | μg |
| Selenium | 10.4 | μg |
| Total solids | 97.0 | g |
| Water content | 3.0 | g |

The amino acid profile of the proteins present in the product is disclosed below:

| | Amount | | Amount |
|---|---|---|---|
| Amino acid | (mg/100 kcal) | Amino acid | (mg/100 kcal) |
| Isoleucine | 104 | Valine | 106 |
| Leucine | 213 | Arginine | 81 |
| Lysine | 179 | Histidine | 45 |
| Methionine | 45 | Alanine | 91 |
| Cystine | 21 | Aspartic acid | 199 |
| Cystathionine | 33 | Glutamic acid | 353 |
| Phenylalanine | 82 | Glycine | 48 |
| Tyrosine | 72 | Proline | 140 |
| Threonine | 97 | Serine | 95 |
| Tryptophan | 38 | Taurine | 8 |

The infant formula also comprises the micronutrients commonly used in the conventional formulae, in the conventional proportions.

### Example 4

### Test Concerning the Physiological Effectiveness of Cystathionine

The main application of cystathionine is to increase the supplies of cysteine which is an amino acid, the need for which is increased in many pathological situations (sepsis, surgical intervention, burns, trauma, etc.). In fact, cysteine is important with respect to protein synthesis, and in particular for the synthesis of proteins of the inflammatory reaction which is activated in situations of attack. These proteins play a major role in the organism's defense and are rich in cysteine.

However, the main target of effectiveness associated with sufficient cysteine supplies is maintenance of the glutathione status. Glutathione is a tripeptide which plays a major role in the organism's defenses in the case of attack (defense against oxidative stress, metabolism-regulating molecule, detoxification of xenobiotics, intracellular reducing agent). Cysteine is the limiting amino acid for the synthesis of glutathione and it has been shown that supplementing the diet with cysteine in rats with sepsis makes it possible to re-establish hepatic glutathione status at the level of a normal animal.

An experimental protocol was therefore carried out in order to test whether, in infected rats (sepsis) which exhibit a hepatic glutathione-depleted status, supplementing the diet with cystathionine is as effective as supplementing with cysteine for re-elevating the glutathione status. Groups of sick rats receiving differentiated diets are compared with regard to the glutathione concentration in various organs but especially in the liver. The liver is in fact the major organ for regulating glutathione synthesis in terms of the whole body. The compared groups received a standard protein diet supplemented either with an amino acid considered to be neutral with respect to glutathione (alanine) (control group), or with cysteine (positive control group), or with cystathionine (test group). A group of animals which are not sick is also realized (normal group). Supplementing with cystathionine makes it possible to maintain the glutathionine concentration in the test group at a level equivalent to that of the positive control group.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A nutritional composition comprising cystathionine.

2. The nutritional composition as claimed in claim 1 wherein said composition is chosen from the group consisting of an enteral nutrition product, a parenteral nutrition product, a food composition for human nutrition and a food composition for animal nutrition.

3. The nutritional composition as claimed in claim 1 wherein the composition comprises between 0.01 and 30 g of cystathionine per liter or per kilo of food composition.

4. A food supplement containing cystathionine.

5. The food supplement as claimed in claim 4 wherein said supplement contains between 0.1 and 99% of cystathionine.

6. The food supplement as claimed in claim 4 wherein said supplement contains between 10 and 50% of cystathionine.

7. A prophylactic composition containing cystathionine.

8. The prophylactic composition as claimed in claim 7 wherein said composition contains between 0.1 and 99% of cystathionine.

9. The prophylactic composition as claimed in claim 7 wherein said composition contains between 30 and 60% of cystathionine.

10. An infant formula containing cystathionine.

11. The infant formula as claimed in claim 10 wherein said formula contains between 0.1 and 99% of cystathionine.

12. The infant formula as claimed in claim 10 wherein said formula contains between 0.1 and 25% of cystathionine.

13. A method of producing a composition comprising the steps of combining cystathionine with fiber.

14. A method for treating a condition selected from the group consisting of:
oxidative stress, hypermetabolism, infection, inflammation, respiratory stress; muscle loss,
preparing, maintaining and following physical exercise; for aiding the recovery of individuals who have suffered burns or who have substantial wounds, and for aiding the development of premature infants or infants with stunted growth; comprising the steps of administering to an individual a therapeutically-effective amount of cystathionine.

15. The method as claimed in claim 14 wherein sufficient cystathionine is provided for maintaining glutathionine status and/or for allowing the synthesis of cysteine-rich proteins.

16. The method of claim 13 wherein the composition is in a form chosen from the group consisting of a nutritional composition, a food supplement, a prophylactic composition, and an infant formula.

17. The method as claimed in claim 13 wherein the composition comprises between 0.01 and 30 g of cystathionine per liter of composition.

18. The method as claimed in claim 13 wherein said composition contains between 0.1 and 99% of cystathionine.

19. The method as claimed in claim 13 wherein said composition contains between 30 and 60% of cystathionine.

20. The nutritional composition as claimed in claim 1 wherein the composition comprises between 0.01 and 30 g of cystathionine per kilo of food composition.
